(19) United States
(12) Patent Application Publication
(10) Pub. No.: US 2025/0268830 A1
(43) Pub. Date: Aug. 28, 2025

(54) SUSTAINED RELEASE INJECTABLE PHARMACEUTICAL FORMULATION OF LEVOTHYROXINE AND PROCESS FOR PREPARATION THEREOF

(71) Applicant: PHARMATHEN S.A., Pallini Attikis (GR)

(72) Inventors: Evangelos KARAVAS, Pallini Attikis (GR); Efthymios KOUTRIS, Pallini Attikis (GR); Lida KALANTZI, Pallini Attikis (GR); Sotiria CHAITIDOU, Pallini Attikis (GR); Nikos LEMONAKIS, Pallini Attikis (GR); Anna PAPADAKI, Pallini Attikis (GR); Vincent BRIEUDES, Pallini Attikis (GR); Artemis KALEZI, Pallini Attikis (GR); Athanasios KATSENIS, Pallini Attikis (GR); Katerina KOTTI, Pallini Attikis (GR)

(73) Assignee: PHARMATHEN S.A., Pallini Attikis (GR)

(21) Appl. No.: 18/697,615

(22) PCT Filed: Oct. 6, 2022
(86) PCT No.: PCT/EP2022/025463

§ 371 Date: Apr. 1, 2024

(30) Foreign Application Priority Data
Oct. 6, 2021 (GR) 20210100683
Dec. 13, 2021 (GB) 2119164.8

(51) Int. Cl. A61K 9/16; A61K 9/00; A61K 9/08; A61K 31/198; A61K 47/34

(52) U.S. Cl. CPC A61K 9/1647; A61K 9/0019; A61K 9/08; A61K 9/1694; A61K 31/198; A61K 47/34

(57) ABSTRACT

A stable sustained release injectable formulation based on poly(D,L-lactide-co-glycolide) microparticles comprising levothyroxine is described. The formulation has a theoretical levothyroxine loading of at least 1.5% w/w. A process for preparing the microparticles is also described. The formulation may be used to control hypothyroidism in adults, congenital hypothyroidism in infants, and acquired hypothyroidism in children.

### TECHNICAL FIELD OF THE INVENTION

The present invention relates to a stable sustained release injectable formulation of levothyroxine or a pharmaceutically acceptable salt, derivative or metabolite thereof, a process for the manufacture of this formulation and the use of the formulation to control hypothyroidism.

### BACKGROUND OF THE INVENTION

Levothyroxine is a hormone and was first isolated in crystalline form in 1915. Its structural formula was discovered in 1926 and it was first synthesized in 1927. The thyroid gland produces hormones that regulate the metabolism of living organisms. Several disorders exist which may lead to the thyroid gland being overactive (hyperthyroidism) or underactive (hypothyroidism), the most common being Hashimoto's disease, Grave's disease, goitre and thyroid nodules. Common hypothyroid symptoms include fatigue, weight gain, inability to tolerate cold, slow heart rate, dry skin and constipation. Levothyroxine in oral form is typically used to treat thyroid hormone deficiency such as hypothyroidism and thyroid tumours and used to treat or prevent goitre, often taking the levothyroxine as a lifelong therapy. For a severe form of thyroid hormone deficiency known as myxedema coma the levothyroxine may be administered intravenously with an initial loading dose, followed by a daily intravenous maintenance dose until the levothyroxine levels are controlled. Intravenous formulations have been used as early as the 1960's.

Currently approved oral and injectable formulations of levothyroxine require daily administration and there is no approved oral or injectable pharmaceutical dosage form of levothyroxine which would sustain the pharmacological effect for a longer period and require less frequent administration (sustained release formulation). Being a drug often taken as lifelong therapy, since hypothyroidism and other thyroid disorders are permanent in most patients, and also considering the necessity of taking it on an empty stomach without other medications, supplements or food for at least half an hour, a reduced frequency of administration would certainly increase patient compliance and contribute to an improvement in life quality. A sustained release injectable formulation would eliminate the need for daily administration and further the need for fasting.

There have been attempts in the past to make injectable formulations with prolonged release. CN 1127634 for example, published in 1996, disclosed controlled release injectable (intramuscular) formulations of estriol, estradiol valearate, testosterone propionate and thyroid—T3 and T4—powder microparticles, said to control the release of the drug for 30 to 90 days. Poly-lactic acid (PLA) was used to form the levothyroxine microparticles. The inventors, however, have not presented any actual release data apart from the exemplary formulations, and there is no approved formulation until today.

Levothyroxine microparticles for topical and transdermal delivery have been prepared in a study published in Biopharm. Drug Dispos. 32:380-388, 2011. Those were prepared in various polymer matrices, i.e. poly D,L lactide (PLA), poly(lactic-co-glycolic acid) (PLGA), poly(N-isopropylacrylamide) (PNIPAM) and ethyl cellulose (EC). The release rates (in vitro) showed that levothyroxine exhibited a burst release irrespective of the polymer type and more than 60% of the drug was released within the first hour. These preparations are clearly not suitable for use in a sustained release injectable formulation aiming at release up to two months (at least a lower burst release would be required in that respect), such a high initial burst rate may cause unpleasant side effects such as restlessness, irritability and nervousness, or serious side effects such as chest pain, palpitations, trouble breathing and heart failure.

There is a need for a sustained release injectable formulation of levothyroxine that has reduced initial burst, controlled release, reduced toxicity, longer body half-life, reduced dosing frequency, enhanced patient compliance, and allows overall reduction of medical care cost.

### SUMMARY OF THE INVENTION

According to the present invention a sustained release pharmaceutical formulation comprising microparticles of levothyroxine or a pharmaceutically acceptable salt, derivative or metabolite thereof with a poly(D,L-lactide-co-glycolide) polymer, wherein the formulation has a theoretical levothyroxine loading of at least 1.5% w/w is provided, which overcomes the deficiencies of the prior art and provides a relatively low initial burst release of levothyroxine and a sustained rate of release over an extended period of time.

The theoretical drug loading (TDL) is calculated using equation:

$$TDL ( \% ) = \frac{Mass of API}{Total Mass of Solids} \times 100$$

It is another object of the present invention to provide a stable parenteral pharmaceutical formulation comprising levothyroxine or a pharmaceutically acceptable salt, derivative or metabolite thereof, as an active ingredient, which is toxicologically safe.

Further object of the present invention is to provide an injectable controlled release formulation comprising levothyroxine or a pharmaceutically acceptable salt, derivative or metabolite thereof, which shows good syringeability, injectability, no clogging or blockage of the syringe needles, good drainage, sterility and re-suspensibility.

A further advantage of the present invention is the formulation of levothyroxine can increase patient compliance to medication and replace the existing treatment regimens which require frequent (daily) oral or injection dosing.

The present invention provides a pharmaceutical formulation for intramuscular or subcutaneous administration, in a single or at multiple injection sites, comprising levothyroxine or a pharmaceutically acceptable salt, derivative or metabolite thereof, as an active ingredient, which is able to offer pharmacological effect for at least a week and up to two months with a single administration. The formulation may be administered from once every month to once every two months, preferably once every month or once every two months.

The present invention further includes sterile injectable controlled release formulations comprising levothyroxine or a pharmaceutically acceptable salt, derivative or metabolite thereof, in the form of ready to use solutions, ready to use suspensions and suspensions/solutions formed by reconstitution with a suitable diluent just before administration (injection).

A further aspect of the present invention provides a fast, simple and cost-effective process for the preparation of a stable injectable pharmaceutical formulation comprising levothyroxine or a pharmaceutically acceptable salt, derivative or metabolite thereof.

Other objects and advantages of the present invention will become apparent to those skilled in the art in view of the following detailed description.

### DETAILED DESCRIPTION

The following terms are used in the present invention with the following meaning:

“Microparticles” or “microspheres” are particles comprising a polymer that serves as a matrix or binder of the particle. The microparticle may contain an active compound dispersed or dissolved in the polymeric matrix and is biodegradable and biocompatible.

“Biodegradable materials” are those that degrade by bodily processes to products readily disposable by the body and should not accumulate in the body.

“Biocompatible” means not toxic to the body, pharmaceutically acceptable, not carcinogenic and which does not significantly induce inflammation in body tissues. “By weight %” means parts by weight per total weight of the microparticle.

“Sustained release formulation”: a pharmaceutical dosage form which provides continuous release of the active pharmaceutical ingredient for a sustained period of time of few days to several months with a single administration.

“PLGA” is poly(lactic-co-glycolic acid) copolymer.

The molecular weight of a synthetic polymer does not have a single value, since different chains will have different lengths and different numbers of side branches.

There will therefore be a distribution of molecular weights, so it is common to calculate the “average molecular weight” of the polymer.

The terms “active agent”, “API”, “active compound”, “drug” are used interchangeably and refer to the pharmacologically active compound of the present invention which is Levothyroxine or it's pharmaceutically acceptable salts, derivatives or metabolites thereof. With the same meaning the term Levothyroxine is used interchangeably throughout the current specification.

“Erosion” is defined as the physical dissolution of a polymer as a result of its degradation.
MeOH: methanol
DCM: dichloromethane
“extraction factor g” is defined as:

$$g = \frac{\begin{matrix}( Total water mass in the final suspension ) * \\ ( solubility of DCM in water )\end{matrix}}{Total mass of DCM in the final suspension}$$

As already mentioned, an object of the present invention is to provide a sustained release injectable formulation of levothyroxine or a pharmaceutically acceptable salt, derivative or metabolite thereof to overcome the issues of the existing thyroid therapies, providing a uniform and constant release rate over an extended period of time.

The formulation comprises levothyroxine or a pharmaceutically acceptable salt, derivative or metabolite levothyroxine and preferably comprises levothyroxine or a pharmaceutically acceptable salt hydrated or anhydrous. Where the formulation comprises a pharmaceutically acceptable salt the salt is selected from sodium, potassium and the like, preferably sodium.

Levothyroxine or it's pharmaceutically acceptable salts, derivatives or metabolites thereof are generally hydrophobic or amphiphilic and insoluble in solvents commonly used to form polymer solutions, thus it is challenging to formulate levothyroxine into matrix polymers. Thus, another object of the present invention is to provide a process for preparing the levothyroxine microparticles of the invented formulation.

A further object of the present invention is to provide a sustained release injectable formulation of levothyroxine or a pharmaceutically acceptable salt, derivative or metabolite thereof with a drug loading suitable to satisfy the maximum dosing requirements.

Yet a further object of the present invention is to provide a sustained release injectable formulation of levothyroxine or a pharmaceutically acceptable salt, derivative or metabolite thereof that exhibits release over an extended period of time, preferably release over a period ranging from 1 week to 2 months, more preferably over a period from 1 to 2 months when injected subcutaneously or intramuscularly while at the same time has no or only a short period passes before release initiation (lag phases) and exhibits low initial burst release compared to the typical values. Lag phases are typically up to 10 days and initial burst release is less than 40% of the total levothyroxine in the microspheres.

In one aspect, the levothyroxine microparticles of the sustained release injectable formulation are manufactured by single emulsion process. According to this process, the PLGA polymer is dissolved in a suitable solvent including, but not limited to dichloromethane, ethyl acetate, tetrahydrofuran, acetonitrile, hexafluoroisopropanol, chloroform or acetone, or a combination thereof to form a PLGA polymer solution. The levothyroxine is dissolved in a suitable solvent, suitable solvents include methanol for the sodium salt form of levothyroxine to form a levothyroxine solution. A dispersed (oil) phase is prepared by mixing the PLGA polymer solution and the levothyroxine solution resulting in a cosolvent system where both PLGA and Levothyroxine are soluble.

According to the present invention a process for preparing a sustained release pharmaceutical formulation for intramuscular or subcutaneous administration, comprising levothyroxine or a pharmaceutically acceptable salt, derivative or metabolite thereof, as an active ingredient, is also provided.

Levothyroxine in the formulations of the present inventions is preferably levothyroxine or a pharmaceutically acceptable salt, more preferably levothyroxine sodium hydrated or anhydrous.

The formulation of the present invention comprises the active pharmaceutical ingredient, a vehicle, other pharmaceutically acceptable excipients and pH adjusting agent(s). The order of mixing the constituents of the formulation is interchangeable.

A general preferred process for the preparation of microparticles comprises the following steps:
PLGA (Poly(D,L-lactide-co-glycolide)) polymer is dissolved under stirring in DCM;
Levothyroxine is dissolved in MeOH and mixed with the polymer solution to form a dispersed phase (DP);
Poly(vinyl alcohol) is dissolved in water for injection at 80° C. The solution is cooled down to 25° C. to form the continuous phase (CP);
The dispersed and the continuous phases are mixed and emulsified using a high shear rotor-stator continuous flow disperser (i.e., in line homogenizer) or an overhead stirrer to form a suspension;
The DP is emulsified in the CP with vigorous stirring;
The suspension is subjected to solvent extraction and evaporation by stirring under controlled temperature at 5-25° C. and air flow to ensure removal of organic solvents and particle solidification;
After 3 hours the microparticles are collected on a glass filter dryer, washed with an excess of water at room temperature and left under vacuum for 24 hours to dry

In a preferred aspect, PLGA is dissolved in dichloromethane under stirring. Levothyroxine sodium is dissolved (under stirring) in methanol and is then added to the polymer solution under stirring to form the dispersed phase. The dispersed phase is kept under a controlled temperature at 5° C. to 25° C., preferably at 5-10° C.

The levothyroxine sodium and PLGA polymer mixture (oil or dispersed phase) formed is subsequently emulsified into an aqueous or continuous phase comprising an aqueous solution of a surfactant, the surfactant is preferably an aqueous solution of polyvinyl alcohol (PVA). In a preferred aspect, levothyroxine sodium and the PLGA polymer mixture is emulsified in a PVA aqueous solution.

The emulsification is preferably performed with a high shear rotor-stator continuous flow disperser (such as in line homogenizer) or an overhead stirrer. The emulsion is then transferred into a receiving tank and remains under controlled temperature, air flow and stirring conditions to remove the organic solvent. The suspension is thermostated at a temperature lower than 20° C., more preferably 5-10° C. for 3 to 4 hours. After the organic solvent is removed a microparticle suspension is formed which is filtered through a glass filter dryer to collect the microparticles with the desired particle size. The microparticles are then washed on the glass filter dryer with an excess of water at room temperature and left under vacuum for 24 hours to dry. The process of the present invention provides microparticles with a particle size distribution of from 10-200 microns as measured by laser light diffraction.

The solubility at 20° C. of the API in MeOH was determined as follows: approximately 50 mg Levothyroxine sodium were weighed in a vial. Methanol was gradually added until the solution became clear. The solubility was determined at 3.41% mass LVX/mass of MeOH.

Subsequently the solubility at 20° C. of PLGA in the mixture DCM/MeOH was examined. A 5% solution of PLGA polymer (PURASORB PDLG 5002A) in DCM was prepared. Methanol was gradually added until polymer precipitation was observed. The minimum allowable DCM/MeOH ratio for a 5% PLGA solution in DCM was determined at 10:3.

A quantity of approximately 0.25 g of polymer and varying quantities of LVX corresponding to theoretical drug loading values 1%, 2%, 3% and 4% were weighed in four vials. A DCM/MeOH mixture at a ratio of 10/3 (to ensure the polymer is always dissolved) was gradually added in each vial. The respective PLGA (PURASORB PDLG 5002A) concentrations, % LVX content and LVX solubility were evaluated and are presented in the table below.

Evaluation of maximum theoretical drug loading value.
DCM/MeOH = 10/3

| | | | DCM + | | | |
|---|---|---|---|---|---|---|
| | | % | MeOH | % LVX | % PLGA | LVX |
| | | TDL | (g) | (in Mix) | (in DCM) | Solubility |
| Vial | PLGA | 1.0 | 1.2 | 0.22 | 27.38 | NO |
| 1 | (g) | | | | | |
| | 0.25251 | 1.0 | 2.5 | 0.11 | 13.01 | Insoluble |
| | | | | | | Qty of LVX |
| | LVX (g) | 1.0 | 3.3 | 0.08 | 9.99 | Almost Clear |
| | 0.00265 | 1.0 | 4.3 | 0.06 | 7.63 | Clear |
| | | 1.0 | 5.5 | 0.05 | 5.92 | Clear |
| Vial | PLGA | 2.0 | 1.3 | 0.39 | 24.93 | NO |
| 2 | (g) | | | | | |
| | 0.25347 | 2.0 | 2.6 | 0.20 | 12.58 | Slurry |
| | LVX (g) | 2.0 | 3.3 | 0.16 | 10.05 | Sluny |
| | 0.00519 | 2.0 | 4.3 | 0.12 | 7.69 | Less Slurry |
| | | 2.0 | 5.6 | 0.09 | 5.92 | Almost Clear |
| Vial | PLGA | 3.0 | 1.3 | 0.59 | 24.74 | NO |
| 3 | (g) | | | | | |
| | 0.25077 | 3.0 | 2.6 | 0.30 | 12.69 | Shary |
| | LVX (g) | 3.0 | 3.3 | 0.24 | 9.97 | Slurry |
| | 0.00771 | 3.0 | 4.2 | 0.18 | 7.70 | Less Shury |
| | | 3.0 | 5.5 | 0.14 | 5.95 | Less Slurry |
| | | 3.0 | 7.7 | 0.10 | 4.25 | Almost Clear |
| | | 3.0 | 9.6 | 0.08 | 3.39 | Almost Clear |
| | | 3.0 | 12.3 | 0.06 | 2.65 | Clear |
| Vial | PLGA | 4.0 | 1.3 | 0.80 | 25.04 | NO |
| 4 | (g) | | | | | |
| | 0.25093 | 4.0 | 2.6 | 0.39 | 12.33 | Slurry |
| | LVX (g) | 4.0 | 3.3 | 0.31 | 9.82 | Shary |
| | 0.01039 | 4.0 | 4.3 | 0.24 | 7.56 | Slurry |
| | | 4.0 | 5.6 | 0.19 | 5.87 | Less Slurry |

The results show that:
levothyroxine is dissolved when the ratio LVX/(DCM+MeOH) is below 0.1%.
for theoretical drug loading higher than 3%, the PLGA concentration will have to be lower than 3% which is too low for the formulation of microparticles.

The results of the solubility study guided the selection of theoretical drug loading. The PLGA (PURASORB PDLG 5002A) concentration was set at 5% in DCM (3.8% in the DCM+MeOH mixture). The theoretical drug loading values in the formulation range between 1.5 and 3%. Such theoretical drug content of at least 1.5%, preferably from 1.5% to 3.0% and more preferably from 2% to 3% and g factor from 1 to 1.5 lead to microparticles with a release of at least one month with low initial lag phase according to the present invention. Levothyroxine microparticles of lower theoretical drug loading do not release for adequate time. Also, PLGA polymers of higher MW and higher lactide-to-glycolide ratio results in a release profile that is considered too slow for the purposes of this invention.

Levothyroxine is preferably dissolved in methanol, preferably when the mass of Levothyroxine to the mass of MeOH is about 3.41%. Additionally, the PLGA is particularly preferred to be dissolved in DCM and the ratio of Levothyroxine to the combined mass of DCM plus MeOH is below 0.1%.

PLGA is a linear aliphatic copolymer obtained at different proportions between its constituent monomers, lactic acid (LA) and glycolic acid (GA). It can be synthesized with any ratio of LA and GA, molecular weights (Mw) with a wide range from below 10,000 up to 200,000 g/mol and in completely amorphous or highly crystalline forms. The amorphous form is found to be suitable for drug release as it provides more even dispersion of a payload in the polymer matrix. The release and degradation rate are profoundly affected by the Mw and the LA/GA ratio. Generally, polymers with higher Mw retain more structural integrity due to cross-linking and exhibit longer release profiles. PLGAs with high GA content are more hydrophilic and allow higher water permeability, resulting in faster degradation rates while at the same time exhibit higher degree of crystallinity.

Nowadays PLGA polymers are commercially available from various sources and easily customizable to the needs of any customer. Exemplary commercially available PLGA polymers that may be used in the present invention are Resomer®, Medisorb®, Expansorb® and Purasorb® PDLG. These polymers are available in a wide range of molecular weights and ratios of lactic acid to glycolic acid, and different polymer chain end groups.

The inventors of the present invention found that PLA used in the prior art sustained release levothyroxine injectable formulation has a slow rate of degradation, leading to a very slow drug release. Surprisingly, it has now been found that the PLGA polymers suitable for the presently claimed levothyroxine sustained release injectable formulations has a lactide to glycolide ratio of from 80:20 to 20:80, more preferably from 75:25 to 25:75 and most preferably from 75:25 to 50:50. Particularly preferred PLGA polymers are those with a lactide to glycolide ratio of 50:50 and 75:25. PLGA polymer average molecular weight (M_{w}) are preferably in ranges between 5 kDa to 200 kDa, more preferably 15-120 kDa. Particularly preferred PLGA polymers are those with average molecular weights of 18 k Da and 115 kDa.

The sustained release injectable formulation of the present invention comprises micropartieles which comprise levothyroxine or a levothyroxine salt, preferably the sodium salt and a polymer, preferably PLGA, is disclosed. Levothyroxine or levothyroxine sodium may be present in a 1.0-5.0 wt % concentration, more preferably 1.5-3.0 wt %, while PLGA concentration may range between 99.0 and 95.0 wt %, preferably 97.0 and 98.5 wt %. Most preferably, levothyroxine is present in a 2.5% concentration and PLGA in a 97.5% concentration.

The PLGA molecular weight is determined by comparison with Gel Permeation Chromatography (GPC) polystyrene (PS) calibration standards (M_{w} range 3-200 KDa). According to the method, the calibration standards and the sample are dissolved in THE and analyzed using two GPC columns with same characteristics connected in row. The retention time values of the standard solutions in combination with the MW value of their CoAs are used to create the calibration curve depicting the retention time vs log(M_{w}). The retention time of the sample is translated to molecular weight based on the calibration curve. The calibration curve is a 3^{rd} order expression of the retention time vs log(M_{w}) calculated by the GPC software.

The in vitro release of the drug from the microparticles of the present invention, is studied in phosphate buffer saline solutions at various pH values. The formulation microparticles are suspended in a phosphate buffer saline release medium and incubated in a water bath system at 37° C. At given time intervals, samples are taken and the amount of drug released is measured by HPLC.

The microparticles of the present invention are reconstituted prior to injection to form a suspension. The suspension comprises the microparticles and a medium (diluent), the medium can be aqueous or non-aqueous. The suspension may also comprise one or more solubilizing or wetting agents, one or more flocculating or suspending agents, one or more antimicrobial preservatives, one or more antioxidants, one or more buffering agents, one or more pH adjusting agents, one or more tonicity adjusting agents, and one or more chelating agents.

Examples of suitable solubilizing agents include diethylene glycol monostearate, diethylene glycol monolaurate, glyceryl monostearate, polyoxyethylene sorbitol beeswax, polyethylene lauryl ether, polyoxyethylene leuryl ether, polyoxyethylene monostearate, polyoxyethylene alkyl phenol, polyethylene sorbitan monooleate, polyethylene sorbitan monolaurate, polyoxyethylene lauryl ether, potassium oleate, sorbitan tristearate, sorbitan monolaurate, sorbitan monooleate, sodium lauryl sulfate, sodium oleate, triethanolamine oleate, etc. Poloxamer and Lutrol® F108 are particularly preferred as solubilizing or wetting agents. Sodium carboxymethylcellulose and hydroxypropylmethylcellulose are particularly preferred as flocculating or suspending agents.

Examples of antioxidants that may also be present include acetone sodium bisulfate, ascorbate, α-tocopherol, bisulfate sodium, butylated hydroxy anisole, butylated hydroxy toluene, cystein, cysteinate HCl, dithionite sodium, gentisic acid, gentisic acid athanolamine, glutamate monosodium, formaldehyde sulfoxylate sodium, metabisulfite potassium, metabisulfite sodium, monothioglycerol, propyl gallate, sulfite sodium, tocopherol alpha, thioglycolate sodium etc. Butylated hydroxyl anisole and/or bisulfite are particularly preferred as antioxidants.

For the preparation of the non-aqueous ready to use controlled release injectable suspension, a buffer is optionally employed. Examples of suitable buffers include: sodium phosphate, potassium phosphate, sodium hydroxide, succinate sodium, succinate disodium, sulfuric acid, tartrate sodium, tartaric acid, tromethamine. Sodium phosphate is particularly preferred as buffering agent.

One or more pH adjusting agents in order to adjust the pH of the suspension from about 6 to about 8, preferably about 7, may be present as well. The pH adjusting agents may be either an acid or a base. Examples of pH adjusting agents suitable for use in the present invention include acetic acid, calcium carbonate, hydrochloric acid, magnesium oxide, magnesium hydroxide, potassium hydroxide, sodium hydroxide etc. Sodium hydroxide and hydrochloric acid are particularly preferred as pH adjusting agents.

For the preparation of the suspension, one or more tonicity adjusting agents is optionally used. Examples of suitable tonicity adjusting agents include magnesium sulfate, maltose, mannitol, polyethylene glycol, polylactic acid, polysorbate, potassium chloride, povidone, sodium chloride, sodium cholesteryl sulfate, sodium succinate, sodium sulfate, sorbitol, sucrose, trehalose. Sodium chloride is particularly preferred as the tonicity adjusting agent.

The suspension may optionally include one or more chelating agents. Examples of suitable chelating agents include calcium disodium ethylenediaminetetraacetic acid (EDTA), disodium EDTA, sodium EDTA and diethylenetriaminepentaacetic acid (DTPA), citric acid, tartaric acid and amino acids, such as lysine and arginine. can also act as chelating agents. Disodium EDTA is particularly preferred as the chelating agent.

The sustained release formulations of the present invention may be used to control hypothyroidism in adults, congenital hypothyroidism in infants and acquired hypothyroidism in children. The formulation may be further used as replacement or supplemental therapy in congenital or acquired hypothyroidism of any etiology, except transient hypothyroidism during the recovery phase of subacute thyroiditis. Specific indications include primary (thyroidal), secondary (pituitary), and tertiary (hypothalamic) hypothyroidism and subclinical hypothyroidism. Primary hypothyroidism may result from functional deficiency, primary atrophy, partial or total congenital absence of the thyroid gland, or from the effects of surgery, radiation, or drugs, with or without the presence of goiter. In another aspect, the formulation may be used in the treatment or prevention of various typed of euthyroid goiters including thyroid nodules, subacute or chronic lymphocytic thyroiditis (Hashimoto's thyroiditis), multinodular goiter and, as an adjunct to surgery and radioiodine therapy in the management of thyrotropin dependent well-differentiated thyroid cancer.

The formulations are preferably administered by subcutaneous or intramuscular injection after being reconstituted with suitable diluent. More particularly formulations may be presented as a kit in which the diluent is packed in a pre-filled syringe and the microparticles are in a vial. Immediately before use the content of the pre-filled syringe (diluent) and the vial (powder) are mixed to prepare the suspension to be injected to the patient. Alternatively, a dual chamber syringe may be used; the microparticles are stored in one chamber of the syringe and the diluent stored in the other chamber of the pre-filled syringe, immediately before injections the contents of each chamber are mixed to form a suspension which is injected to the patient.

Suitable diluents include inactive ingredients such as carboxymethylcellulose sodium, mannitol, sodium chloride, sodium hydroxide, polysorbate, acetic acid, sodium dihydrogen phosphate monohydrate, disodium phosphate heptahydrate.

The formulations are preferably administered once every one or two months.

### EXAMPLES

### Example 1 (Comparative Example)

Microparticles were prepared with the process as exemplified in the description above using three types of PLGA polymers, PURASORB PDLG 5002A with 18 kDa molecular weight and RESOMER RG 504H with 60 kDa molecular weight, both having lactide to glucolide ratio 50:50 and PURASORB PDLG 7510 with 115 kDa molecular weight and lactide to glucolide ratio 75:25.

These were characterized in terms of drug loading, particle size distribution and in vitro release. The results are shown in the table below.

PSD (particle size distribution) and in vitro release
rate of formulations LVX_1.2, LVX_1.3, LVX_1.4

| | Formulation | | | | | | |
|---|---|---|---|---|---|---|---|
| | Parameter | Quality Attributes | | | | | |
| Form- | tested | Particle Size | | | | | |
| ulation | PLGA type | Distribution (μm) | | | % Release/Days | | |
| name | (MW) | Dᵥ(10) | Dᵥ(50) | Dᵥ(90) | 10% | 50% | 80% |
| LVX 1.2 | 5002A (18 kDa) | 27.9 | 57.7 | 103.7 | 0.03 | 12.5 | 17.7 |
| LVX 1.3 | 504H (60 kDa) | 35.1 | 79.9 | 140.6 | 15.0 | 27.7 | 32.6 |
| LVX 1.4 | 7510 (55 kDa) | 44.7 | 87.6 | 181.8 | 0.3 | — | — |

Formulations LVX_1.2 and LVX_1.3 exhibit sigmoidal profiles with lag phases of ˜10 and 20 days respectively followed by a main release phase up to ˜day 25 and day 40 respectively.

The results reveal the effect of polymer MW on the PSD and the release profile of the levothyroxine microparticles. Higher MW (formulation LVX_1.3) led to larger microparticles (higher viscosity of the dispersed phase during emulsification) and consequently to a slower release profile (lower surface area per unit volume leading to a reduced rate of water permeation and matrix degradation).

In the case of formulation LVX_1.4, in addition to the high MW, a higher lactide-to-glycolide ratio (75:25 as opposed to 50:50 in the other two formulations) resulted in a release profile that is considered too slow for the purposes of this invention. The lactide-to-glycolide ratio affects the degradation rate of the microparticles and consequently the release rate.

### Example 2

Two formulations (LVX_1.5 and LVX_1.6) were prepared based on LVX_1.2 and one (LVX_1.12) based on LVX_1.3, introducing changes in the theoretical drug loading and the g factor. Both parameters are increased (theoretical drug content from 1.5% to 2.0 and 3.0% and g factor from 1 to 1.5) aiming at a higher drug content.

Theoretical Drug loading (TDL), PSD and in vitro release
rate of formulations LVX_1.5, LVX_1.6 and LVX_1.12(

| | Process | | Quality Attributes | | | | | |
|---|---|---|---|---|---|---|---|---|
| Form- | parameters | | Particle Size | | | % Release/ | | |
| ulation | Theoretical | | Distribution (μm) | | | Days | | |
| name | DL (%) | g | Dᵥ(10) | Dᵥ(50) | Dᵥ(90) | 10% | 50% | 80% |
| LVX_1.5 | 3 | 1.0 | 27.4 | 52.3 | 92.3 | 0.04 | 21.5 | 29.6 |
| LVX_1.6 | 2 | 1.5 | 24.7 | 49.7 | 91.7 | 0.04 | 19.7 | 27.0 |
| LVX_1.12 | 2.5 | 1.0 | 31.7 | 64.5 | 138.2 | 0.0 | 28.5 | 48.0 |

The increase in the theoretical drug loading and the g factor values led to higher actual drug content. In addition to this, the release window of both formulations (LVX 1.5 and 1.6) became longer compared to the LVX_1.2 formulation. More specifically, the time point of 50% release is moved from 14 days (LVX 1.2) to 20-22 days (LVX 1.5 and LVX 1.6) and that of 100% release from 25 days to 35 days respectively. The observed shift to longer release profiles is justified by the increase in the core drug load. Similar initial burst values were obtained suggesting that the applied g values in the range of 1.0-1.5 result in similar ratios of surficial and sub surficial drug content to total drug content.

Similar to what was observed for the low MW polymer PLGA, the increase in drug loading leads to longer release profiles for the high MW. The increase in drug loading seems to also correlate with an increase in initial burst (compare LVX_1.3 with LVX_1.12).

The formulation trials performed with lower MW (18 kDa) and higher MW around 55 kDa PLGAs, exhibit a release window ranging from 25 to 35 days or up to 60 days, respectively. Formulations with high MW polymer also exhibit lower initial burst.

1. A sustained release pharmaceutical formulation comprising microparticles of levothyroxine or a pharmaceutically acceptable salt, derivative or metabolite thereof with poly(D,L-lactide-co-glycolide) polymer, wherein the formulation has a theoretical levothyroxine loading of at least 1.5% w/w.

2. The pharmaceutical formulation according to claim 1, wherein the formulation has a theoretical levothyroxine loading of 1.5% w/w to 3% w/w.

3. The pharmaceutical formulation according to claim 1, wherein the formulation has a theoretical levothyroxine loading of 2% w/w to 3% w/w.

4. The pharmaceutical formulation according to claim 1, wherein the poly(D,L-lactide-co-glycolide) polymer has a ratio of lactide to glycolide of from 80:20 to 20:80.

5. The pharmaceutical formulation according to claim 1, wherein the poly(D,L-lactide-co-glycolide) polymer has a ratio of lactide to glycolide of from 75:25 to 25:75.

6. The pharmaceutical formulation according to claim 1, wherein the poly(D,L-lactide-co-glycolide) polymer has a ratio of lactide to glycolide of from 75:25 to 50:50.

7. The pharmaceutical formulation according to claim 1, wherein the poly(D,L-lactide-co-glycolide) polymer has a ratio of lactide to glycolide of 50:50.

8. The pharmaceutical formulation according to claim 1, wherein the poly(D,L-lactide-co-glycolide) polymer has a ratio of lactide to glycolide of 75:25.

9. The pharmaceutical formulation according to claim 1, wherein the polymer has a weight average molecular weight in a range of from 5 to 200 kDa.

10. The pharmaceutical formulation according claim 1, wherein the polymer has a weight average molecular weight in a range of from 15 to 120 kDa.

11. The pharmaceutical formulation according to claim 1, wherein the polymer has a weight average molecular weight in a range of from 18 to 115 kDa.

12. The pharmaceutical formulation according to claim 1, wherein the microparticles have a particle size in a range of 10 to 200 microns as measured by laser light diffraction.

13. The pharmaceutical formulation according to claim 1, which is reconstituted with a diluent before intramuscular or subcutaneous administration.

14. The pharmaceutical formulation of claim 13, wherein the diluent comprises at least one selected from the group consisting of carboxymethylcellulose sodium, mannitol, sodium chloride, sodium hydroxide, polysorbate, acetic acid, sodium dihydrogen phosphate monohydrate, and disodium phosphate heptahydrate.

15. The pharmaceutical formulation of claim 1, which is administered by intramuscular or subcutaneous injection.

16. The pharmaceutical formulation of claim 1, which is administered from once every month to once every two months.

17. The pharmaceutical formulation of claim 1, which is administered once every month or once every two months.

18. The pharmaceutical formulation of claim 1, which is reconstituted with a diluent and administered intramuscularly or subcutaneously with a dual chamber syringe or a kit having a syringe pre-filled with the diluent and a separate vial containing the microparticles.

19. The pharmaceutical formulation of claim 1, further comprising levothyroxine or a pharmaceutically acceptable salt.

20. The pharmaceutical formulation of claim 1, further comprising levothyroxine sodium hydrated or anhydrous.

21. A method of controlling hypothyroidism in an adult, congenital hypothyroidism in an infant, or acquired hypothyroidism in a child, the method comprising:
administering the formulation of claim 1 to the adult, the infant, or the child.

22. A method for preparing the microparticles of levothyroxine present in the formulation of claim 1, the method comprising:
dissolving poly(lactic-co-glycolic acid) copolymer under stirring in dichloromethane to produce a polymer solution;
dissolving levothyroxine in methanol and mixing with the polymer solution to form a dispersed phase;
dissolving poly(vinyl alcohol) in water at 80° C. and cooling to 25° C. to form a continuous phase;
mixing and emulsifying the dispersed phase and the continuous phase with a high shear rotor-stator continuous flow disperser or an overhead stirrer to form a suspension;
stirring the suspension at a temperature in a range of 5° C. to 25° C. and under controlled air flow for at least 3 hours to remove organic solvents and solidify the microparticles by solvent extraction and evaporation; and
collecting the microparticles on a glass filter dryer, washing with an excess of water at room temperature, and drying under vacuum for 24 hours.

23. The method of claim 22, wherein the dispersed phase is maintained at a temperature in a range of 5° C. to 25° C.

24. The method of claim 22, wherein the dispersed phase is maintained at a temperature in a range of 5° C. to 10° C.

25. The method of claim 22, wherein a mass of levothyroxine to a mass of methanol is below 3.41%.

26. The method of claim 22, wherein a ratio of levothyroxine to a combined mass of dichloromethane plus methanol is below 0.1%.
